(19) United States
(12) Patent Application Publication
(10) Pub. No.: US 2016/0012587 A1
(43) Pub. Date: Jan. 14, 2016

(54) SYSTEM AND METHOD FOR NON-INVASIVE DETERMINATION OF CARDIAC ACTIVATION PATTERNS

(71) Applicant: Quynh A. TRUONG, Boston, MA (US)

(72) Inventor: Quynh A. Truong, Boston, MA (US)

(21) Appl. No.: 14/772,099

(22) PCT Filed: Feb. 24, 2014
(86) PCT No.: PCT/US14/17914

§ 371 Date: Sep. 2, 2015

Related U.S. Application Data
(60) Provisional application No. 61/773,510, filed on Mar. 6, 2013.
(60) Provisional application No. 61/773,484, filed on Mar. 6, 2013.

(51) Int. Cl. G06T 7/00; A61B 6/00; G06T 7/20; A61B 6/03

(52) U.S. Cl. CPC G06T 7/0014; A61B 6/032; A61B 6/463; A61B 6/503; A61B 6/5217; A61B 6/5288; G06T 7/2006; A61B 2576/023; G06T 2207/10081; G06T 2207/20112; G06T 2207/10024; G06T 2207/20221; G06T 2207/30048

(57) ABSTRACT

A system and method for determining a pattern of activation of a heart of a subject. An imaging dataset is acquired of a portion of the subject including the heart and the imaging dataset is processed=to identify a motion parameter of the heart. The motion parameter of the heart is mapped over time to create a pattern of activation of the heart. A global LV dyssynchrony index is automatically generated and analyzed using changes in wall thickness of the heart over time. A report is generated indicating the pattern of activation of the heart of the subject.

### CROSS-REFERENCE TO RELATED APPLICATIONS

The present application is based on, claims priority to, and incorporates herein by reference in its entirety, U.S. Provisional Application Ser. No. 61/773,484, filed Mar. 6, 2013, and entitled “SYSTEM AND METHOD FOR NON-INVASIVE DETERMINATION OF CARDIAC ACTIVATION PATTERNS,” and U.S. Provisional Application Ser. No. 61/773,510, filed Mar. 6, 2013, and entitled “AUTOMATED GLOBAL CT DYSSYNCHRONY INDEX.”

### STATEMENT REGARDING FEDERALLY SPONSORED RESEARCH

N/A

### BACKGROUND OF THE INVENTION

The present invention relates to systems and methods for analyzing cardiac function. More particularly, the invention relates to a method for determining a pattern of activation of a subject's heart and analyzing global left ventricular (LV) dyssynchrony using a combined cardiac anatomy and motion dataset.

Heart failure is one of the leading causes of morbidity and mortality in the United States. The main therapeutic goals to combat heart failure are focused on reducing major adverse cardiac events (MACE) and mortality, alleviating symptoms, and improving functional status. However, despite substantial poly-pharmacological therapies, many patients experience refractory heart failure symptoms. Cardiac resynchronization therapy (CRT) is a device therapy that has gained worldwide acceptance as adjuvant treatment for patients with refractory heart failure, left ventricular (LV) systolic dysfunction, and wide QRS duration. CRT systems build upon pacing systems to also synchronize the function of the heart. Specifically, in addition to pacing the right ventricle, an extra LV lead is typically placed via the coronary sinus into a branch of the coronary veins to pace the LV and “synchronize” the heart. Thus, CRT therapies require invasive deployment of the CRT system.

Unfortunately, about 1 out of 3 patients who receive CRT therapy do not demonstrate clinical improvement. Factors leading to high non-response rate include, for example, suboptimal LV lead placement, intraventricular dyssynchrony, and myocardial scar. Intraventricular (or LV) dyssynchrony occurs when there is delayed electromechanical activation within regions of the left ventricle that result in discordant and inefficient contraction. Given that CRT is expensive, invasive, and carries a procedural risk, improvement of CRT by optimal device implantation is warranted. It is postulated that pacing over the site with maximal discordance and avoiding a region of myocardial scar may result in a better outcome. Additionally, proper patient selection for CRT by examining the extent of dyssynchrony and/or myocardial scar is desirable to reduce the number of unbeneficial implants and provide patients with realistic expectations,

Despite these indications, currently, the vast majority of CRT implantation is performed without pre-procedural imaging of the coronary venous anatomy. At the time of CRT implantation, the LV lead is placed into one of the coronary veins under fluoroscopic guidance. This is performed irrespective of the patient's anatomy and without knowledge of the site of most delayed activation or myocardial scar. If there is nota good vein to target lead placement, then epicardial pacing may be an alternative approach that is used to remediate the failed primary deployment strategy. Thus, pre-operative information on coronary venous anatomy by noninvasive means may be useful in determining the implantation strategy. To provide end-stage heart failure patients with a chance for clinical improvement and to reduce excessive burden and cost on an individual and societal level, a personalized approach to CRT is necessary. Specifically, the LV lead placement should be optimized to target the site of latest activation and avoid areas of myocardial scar.

Imaging of the coronary veins by cardiac computed tomography (CT) prior to CRT implantation is feasible and deemed as “appropriate” in many clinical settings. Neither echocardiography nor nuclear cardiology scans have adequate spatial resolution to allow for coronary venous assessment. Unfortunately, CT imaging, while well suited to anatomical imaging and providing functional information from anatomical images acquired over time, is currently ill-suited to provide physiological information. In contrast, electroanatomical mapping (EAM) allows measurements of both myocardial electrical activation and myocardial voltage. As the heart is electrically activated (QRS complex), the activation of the LV myocardium starts at the level of the septum, spreading to the apex and then to the base of the heart. With EAM, a color-coded activation map can illustrate areas of delayed activation over time. However, unlike CT imaging, EAM unfortunately is highly invasive. This catheter-based approach also enables the assessment of a myocardial voltage map, whereby low voltage areas less than about 1.5 mV represent scar. Hence, myocardial scar can be differentiated from non-scarred myocardium by voltage maps at areas of delayed activation on EAM. Both maps are performed with simultaneous intracardiac and surface electrocardiography (ECG). However, EAM is not clinically indicated or performed to guide CRT implantation due to its invasiveness and prolongation of device implantation time.

Therefore, there is a need for systems and methods to non-invasively provide salient information that can be used to assist in planning and implementing CRT procedures to improve clinical outcomes as well as to determine candidates that will receive substantial benefits from CRT from those that will not.

### SUMMARY OF THE INVENTION

The present invention overcomes the aforementioned drawbacks by providing a system and method that noninvasively determines cardiac activation patterns with potential to identify the site of latest activation to guide LV lead placement for device therapy. In particular, the present invention can use cardiac imaging data and velocity as a surrogate for electrical activation by determining the cardiac mechanical activation patterns and other information about a subject that can be used to plan or improve implementation of procedures, such as CRT deployment, and automatically analyze global LV dyssynchrony using changes in wall thickness of the LV over time.

In accordance with one aspect of the invention, a system for determining a pattern of activation of a heart of a subject is disclosed. The system includes a memory having stored thereon an imaging dataset acquired from a portion of the subject including the heart. The system further includes a processor having access to the memory and the imaging dataset stored thereon and configured to process the imaging dataset to identify a motion parameter and map the motion parameter over time to create a pattern of activation of the heart of the subject over time. A display coupled to the processor and configured to display the pattern of activation of the heart in a series of images of the heart of the subject over time.

In accordance with another aspect of the invention, a method for determining a pattern of electro-mechanical activation of a heart of a subject is disclosed. The method includes acquiring an imaging dataset from a portion of the subject including the heart and segregating the imaging dataset into a cardiac anatomy dataset and a motion dataset. The cardiac anatomy dataset is then processed to identify a cardiac phase of the heart over time, and the motion dataset is processed to identify a motion parameter to operate as a surrogate for electrical activation. The cardiac anatomy dataset and the motion dataset are then merged together to form a combined dataset. A report is generated related to the pattern of electro-mechanical activation of the heart of the subject using the combined dataset.

In accordance with another aspect of the invention, a method for determining a pattern of activation of a heart of a subject is disclosed. The method includes acquiring an imaging dataset from a portion of the subject including the heart and processing the imaging dataset to identify at least one motion parameter of the heart. The motion parameter is then mapped over time to create a pattern of activation of the heart of the subject over time. The pattern of activation of the heart of the subject over time is then displayed.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a block diagram of a system configured to implement the present invention.

FIG. 2 is a flow chart setting forth the steps of processes for creating a cardiac activation map in accordance with the present invention.

FIG. 3 is a diagram illustrating one implementation of the process described with respect to FIG. 2 using a computed tomography (CT) dataset and velocity motion parameter.

FIG. 4 is a set of images showing an electrical activation pattern from cardiac septum to apex-base acquired with EAM and mechanical activation pattern maps derived using the present invention.

FIG. 5 is a flow chart setting forth the steps of processing for automatically generating a dyssynchrony index in accordance with the present invention.

FIG. 6 is an image showing short axis slices with endocardial and epicardial casts of the left ventricle.

FIG. 7 is a resultant short axis image obtained from one of the short axis slices of FIG. 6.

FIG. 8 is a graph showing wall thicknesses of six standardized segments of the LV myocardium over 1 cardiac cycle at a single ventricular slice level from a normal subject.

FIG. 9 is a graph showing wall thicknesses of six standardized segments of the LV myocardium over 1 cardiac cycle at a single ventricular slice level from a subject with heart failure.

### DETAILED DESCRIPTION OF THE INVENTION

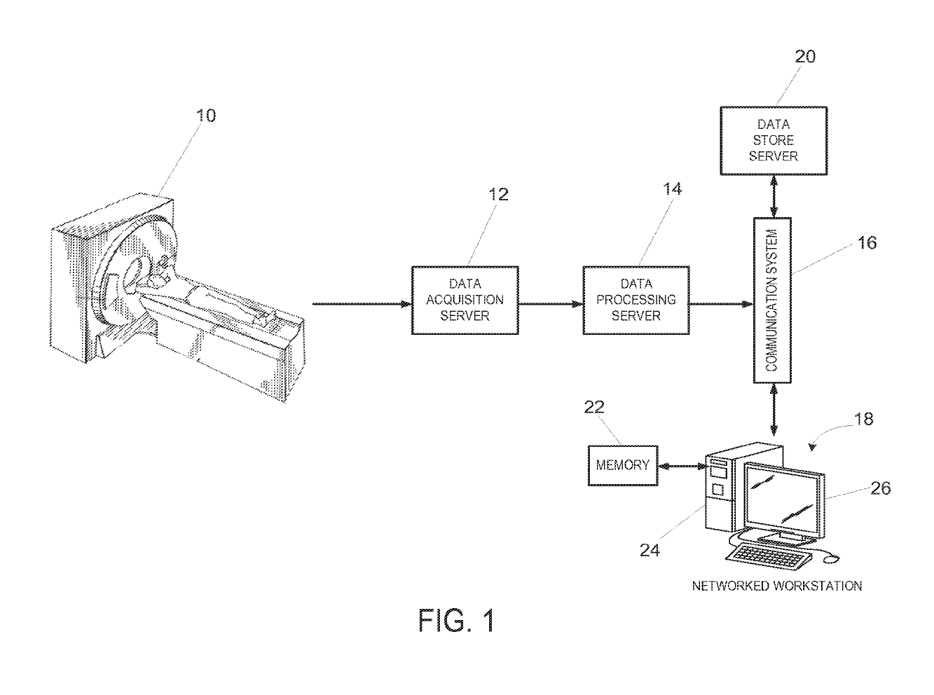

Referring particularly now to FIG. 1, a system 10 is shown that is configured to acquire a raw imaging data from a subject being imaged. The raw data may be, for example, computed tomography (CT) data acquired by a CT imaging system, such as illustrated in FIG. 1; however, other imaging modalities may also be used to acquire the imaging data, such as magnetic resonance imaging (MRI) systems and other imaging systems. For exemplary purposes and to be consistent with the example system that is illustrated in FIG. 1, reference will be made to a CT dataset; however, other dataset types may be likewise utilized. The raw dataset is sent to a data acquisition server 12 coupled to the system 10. The data acquisition server 12 then converts the raw data to a dataset suitable for processing by a data processing server 14, for example, to reconstruct one or more images from the dataset. The dataset or processed data or images can then be sent over a communications system 16 to a networked workstation 18 for processing or analysis and/or to a data store server 20 for long-term storage. The communication system 16, which may be local or wide, a wired or wireless, network including, for example, the internet, allows the networked workstation 18 to access the data store server 20, the data processing server 14, or other sources of information.

The networked workstation 18 includes a memory 22 that can store information, such as the dataset. The networked workstation 18 also includes a processor 24 configured to access the memory 22 to receive the dataset or other information. The network workstation 18 also includes a user communication device, such as a display 26, that is coupled to the processor 24 to communicate reports, images, or other information to a user.

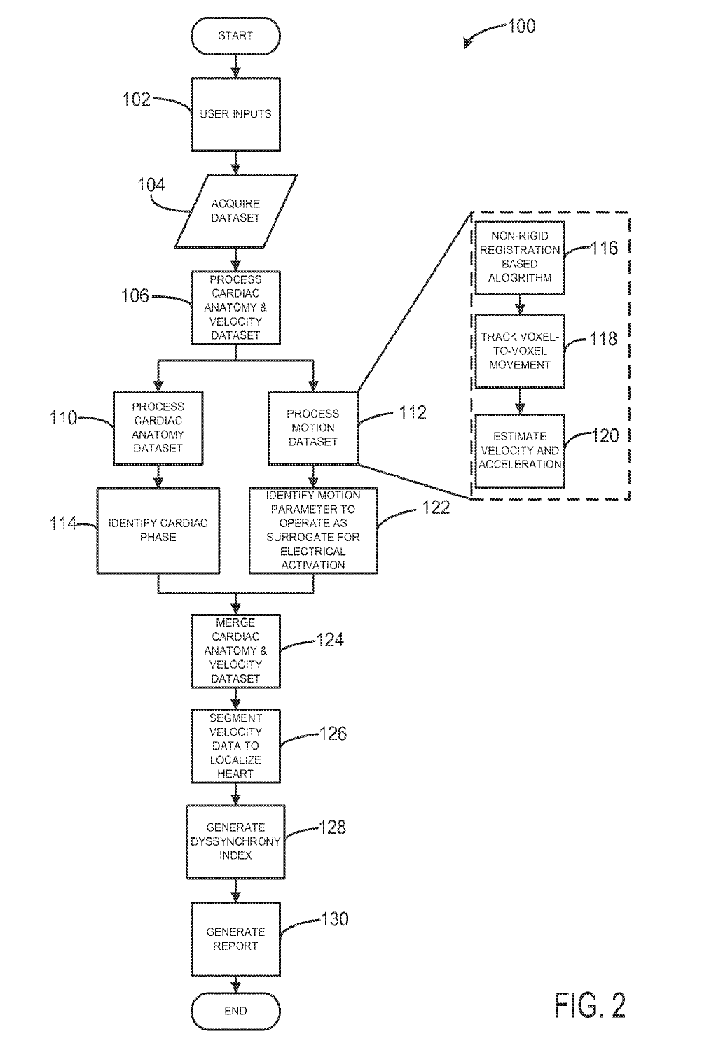

Referring now to FIG. 2, a flow chart setting forth exemplary steps 100 for processing datasets to derive cardiac activation information about a subject non-invasively is provided. To start the process, user inputs 102 are entered into the networked workstation 18 by a user, as shown in FIG. 1. The user inputs can include patient demographics, a patient's previous medical history, and the like. A dataset, for example, an imaging dataset, such as a CT dataset, is then acquired at process block 104. The imaging dataset may be acquired with a CT system, or other imaging system or dataset about the subject's heart. For example, the dataset may be derived using a magnetic resonance imaging (MRI) system or other imaging or non-imaging systems, preferably, to acquire the dataset non-invasively. In the example of a CT dataset, a clinician may operate an imaging system, such as the system 10 of FIG. 1, to acquire a dataset specifically for this process, or a previously-acquired dataset that includes a portion of the patient's heart may be accessed.

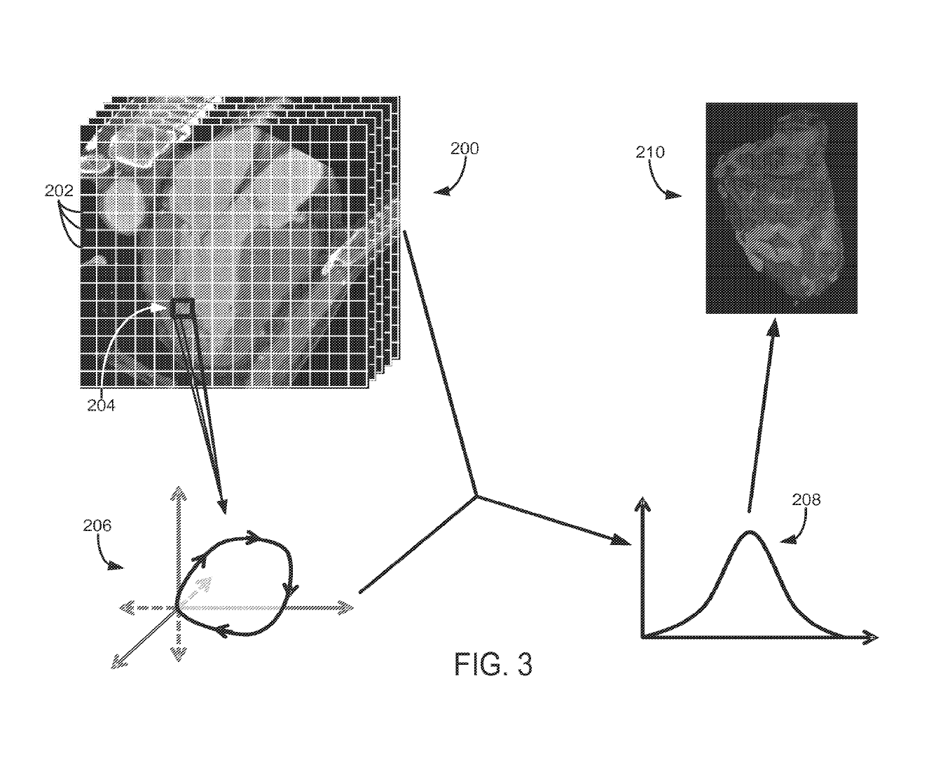

In this regard, the dataset includes information about the cardiac anatomy and motion of the cardiac anatomy over time. The dataset, may include raw imaging data that is then reconstructed or previously-reconstructed images. For example, referring to FIG. 3, the dataset may be reconstructed into a time-series of images 200. At process block 106, the dataset is processed. As will be described, the processing can be conceptualized as processing an anatomy dataset and a motion dataset. Though illustrated in parallel, the following processing may be performed in parallel or series. Specifically, processing cardiac anatomy at process block 110 and processing motion at process block 112 may be performed in series or parallel. As will be described, the processes, regardless of implementation preferences, results in the identification of cardiac phase at process block 114 and motion parameters that, as will be described, can serve as a surrogate for electro-mechanical activation in clinical planning procedures at process block 122. For example, the motion parameter may be velocity or, more specifically, a time-to-first-peak systolic velocity may be identified.

As illustrated in FIG. 3, the time-series of images 200 may be processed using a spatial analysis grid 202, which in FIG. 3 is, for illustration purposes, not to scale. The time-series of images 200 are processed using a motion detection algorithm to help track the anatomy for purposes of determining cardiac phase and identifying motion parameters. For example, a finite element method (FEM) may be used to track motion. Specifically, a spatial vector of translation of a target region 204 throughout the cardiac cycle 206 can be determined, which yields a dataset from which cardiac phase can be determined. Likewise, such motion detection can be used to derive a scalar of three dimensional movement over time to determine an individual target region's 204 movement relative to an initial cardiac phase, to thereby determine a relative peak 208. As will be described, this tracking of the cardiac cycle and determination of motion, can be used to create an activation map registered to anatomical images 210.

In one example, the anatomical images, such as described above, may be processed at 5 percent increments of the R-R interval (20 phases) to identify the cardiac phases at process block 114. For example, the end of the systole and diastole cardiac phases of the cardiac cycle may be identified and tagged within the images. At process block 112, the motion dataset may be processed to determine and track a predetermined motion parameter over time. For example, the processing of the motion dataset at process block 112, in one example, may include using a non-rigid registration (60 phases) algorithm, as indicated at process block 116, and tracking voxel-to-voxel movement throughout the cardiac cycle, as indicated at process block 118, in order to yield an estimated velocity and acceleration at process block 120, which can be tracked throughout ventricular systole and diastole. The non-rigid registration algorithm may be designed to align the cardiac structures from phase-to-phase, virtually tracking individual voxels through the cardiac cycle. Further, by applying physics modeling, parameters such as velocity can be calculated, such that during cardiac contraction and relaxation, myocardial velocity depicts the distance the myocardium has moved over time (mm/sec) and acceleration as velocity over time (mm/sec²).

The ability to track such movement on voxel-by-voxel is related to voxel size. Thus, it is contemplated that, at process block 102, the user may specify a desired voxel size. However, it is also contemplated that a predetermined voxel size may be utilized. Notably, the choice of voxel size is related to the noise associated with tracking the motion parameter. For example, increased voxel size aids in reducing noise. In implementation, this constraint can be managed using a designation of center voxel and adjacent voxels to form a voxel cube. For example, a small volume of interest (VOI) can be selected using a single voxel with one adjacent voxel on either side of the center voxel to create an overall VOI designated as a kernel setting of K1V3, which would include nine voxels. A larger voxel cube could have, for example, a center voxel with 3 adjacent voxels on each side for a total voxel length of 7 voxels, which would include 49 voxels and be designated as a kernel setting of K3V7. Using this construct, any of a variety of voxel sizes can be created. Applying the above concepts, an example dataset may include 3540 slices over 20 series. This dataset might be processed at a kernel setting of K3V7 to include 21×21×21 voxels or at larger kernel settings. The process might analyze every voxel at every phase.

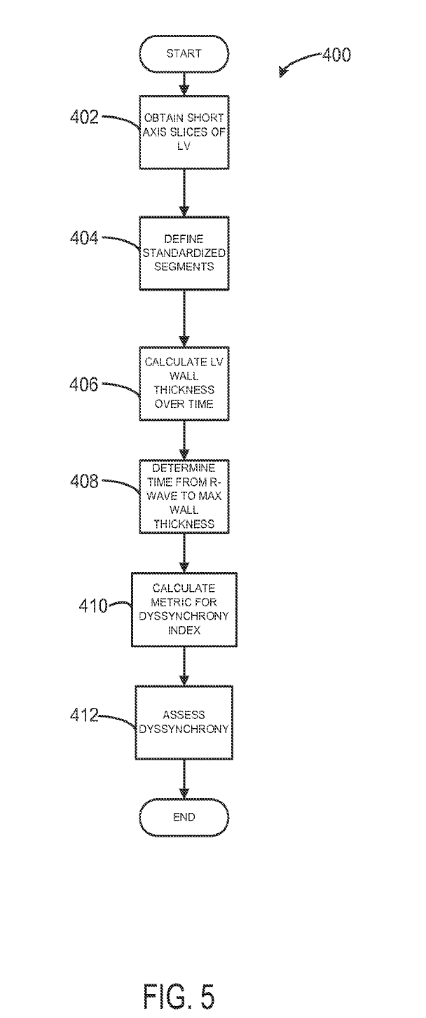

Once the cardiac phases and motion parameter operating as a surrogate for electro-mechanical activation are identified, the cardiac anatomy data (images) and motion dataset (tracked motion parameters) can be merged at process block 124. In doing so, at process block 126, the anatomic dataset may be used to mask or segment the motion dataset to localize the motion dataset to the heart and superimpose a deformable color kinematic/velocity map over the cardiac anatomy datasets. For example, the system may segment anatomical images by identifying the LV myocardium, such as using contours of the endocardium and epicardium. Additionally, at process block 128 a dyssynchrony index may be generated, as will be described in more detail below with respect to FIG. 5, to analyze global LV dyssynchrony.

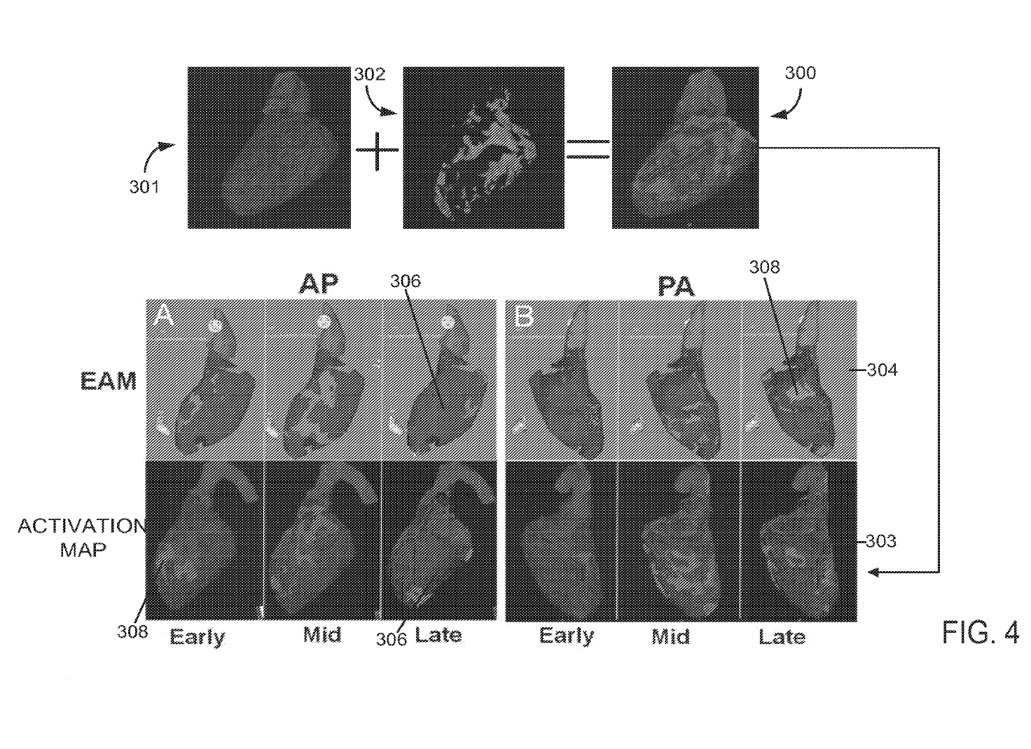

At process block 130 a report is generated based on the preceding analysis. For example, maps of the motion parameter over time can be used to create a pattern of mechanical activation of the heart of the subject over time 300, as shown in FIG. 4. For example, the report may include the activation map 300, which includes information about the anatomy of the heart 301, as well as regions that are mechanically activated at that given time 302. The one activation map 300 is a representation of mechanical activation at a given time. Accordingly, the generated report can include a plurality of images and/or videos 303 of movement of myocardial segments throughout the cardiac cycle displayed as parametric maps overlaying volume rendered images, such that patterns of myocardial electrical activation conduct to similar patterns of mechanical motion due to effects of electromechanical coupling. Electromechanical coupling is a process that links electrical cardiac excitation to mechanical contraction of the myocardium. Therefore, the above-described process can be used to map contractility to reflect the activation pattern seen on electroanatomical map (EAM) 304.

Thus, the above-described report may include deformable color kinematic map that uses a binary color template, shown in FIG. 4, such that myocardial regions of a first color 306 (e.g., blue) become a second color 308 (e.g., red) after the first upslope curve in the cardiac cycle is reached. The propagation pattern is visualized as the conversion of a region from the first color 306 to the second color 308 at a particular time point, after that time point the second color 308 converts back to the first color 308. A region can only become the second color 308 at one time point, while that same region is the first color 306 at all other time points.

The above-described process, as a non-limiting example, may be embodied as kinematics software to track myocardial time-to-first-peak systolic velocity and display the tracked information in a binary color scheme that reflects a myocardial activation pattern, similar to an invasive electroanatomical map (EAM) 304. The use of the post-processing kinematics software to assess myocardial velocity and acceleration provides a non-invasive imaging tool with potential widespread clinical applications. The strength of cardiac CT lies in the ability to clearly demonstrate anatomy, however at present evaluation of myocardial function is limited to gray-scale images. Myocardial velocity and acceleration measured on along the longitudinal, radial and circumferential directions of the LV provides the ability to quantitatively assess, for example, LV global and regional myocardial contractility. Moreover, integration of cardiac anatomy with function by superimposing velocity color maps onto, for example, gray scale CT images provides previously-unavailable information to clinicians without the need for interventional procedures.

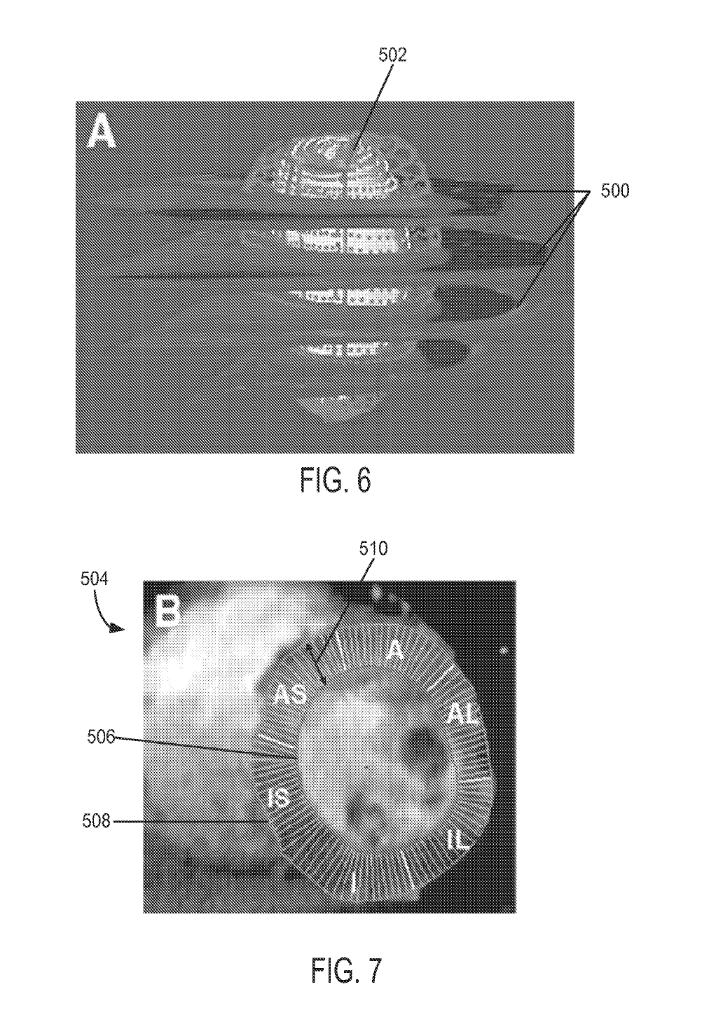

Turning now to FIG. S a flow chart setting forth exemplary steps 400 for automatically generating a dyssynchrony index is provided, as previously mentioned with respect to process block 128 of FIG. 2. The dyssynchrony index may be, for example, a CT global dyssynchrony index that that uses a dyssynchrony metric based on wall thickness of the LV and eliminates the requirement of manual tracing of the endocardial and epicardial boundries, thus reducing the time of post-processing. To start the process, short axis slices with endocardial and epicardial casts of the LV may be obtained at process block 402 using multidetector CT (MDCT) imaging, for example. Exemplary short axis slices 500 of a left ventricle 502 obtained using MDCT are shown in FIG. 6, and a resultant short axis image 504 obtained from one of the short axis slices is shown in FIG. 7.

At process block 404 of FIG. 5, standardized segments of the LV are defined. The standardized segments may be segments of the LV myocardium and may include, but are not limited to, the anterior, anterolateral, anteroseptal, inferior, inferolateral, and inferoseptal segments. The standardized segments may be defined at process block 404 by utilizing a software program configured to trace the endocardial and epicardial boundaries of the LV. As shown in FIG. 7, the endocardial boundary 506 and epicardial boundary 508 of the short-axis image 504 are segmented into six standardized segments, namely A (anterior), AL (anterolateral), IL (inferolateral), I (inferior), IS (inferoseptal), and AS (anteroseptal).

Once the standardized segments of the LV are defined at process block 404, a wall thickness at each of the standardized segments of the LV can be calculated over time at process block 406. LV wall thickness may be depicted as a radial distance 510 between the endocardial boundary 506 and epicardial boundary 508, as shown in FIG. 7. Because the dyssynchrony metric is based on wall thickness that uses both endocardial and epicardial boundaries, the wall thickness analysis may allow for a more precise assessment of differences in wall mechanics and myocardial contractile force, allowing for comprehensive assessment of dyssynchrony.

Returning to FIG. 5, the time from R-wave to maximal wall thickness may be determined at process block 408 for each of the six standardized segments for all slices, for example. Then, as process block 410, a metric, such as standard deviation (SD) of the time-to-maximal wall thickness of the six segments per slice averaged for all slices, may be calculated to define the global dyssynchrony index. Once the metric is calculated, the dyssynchrony may be assessed at process block 412. For example, more variability between the times to maximal wall thickness of each segment may reflect a greater degree of dyssynchrony, and more uniformity between time to maximal wall thickness of each segment may reflect a lesser degree of dyssynchrony.

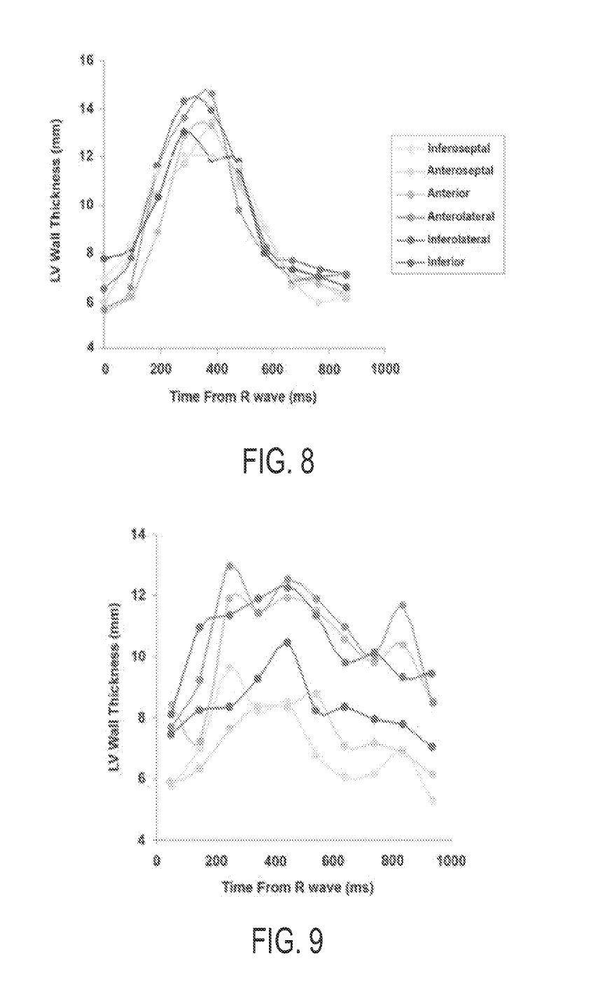

Referring now to FIGS. 8 and 9, time-to-maximal LV wall thickness graphs are shown from a normal subject and a subject with heart failure, respectively. The graphs display the wall thickness of the six standardized segments of the LV myocardium over 1 cardiac cycle at a single ventricular slice level. As shown in FIG. 9, for example, the different segments from the heart failure subject with dyssynchrony contract non-uniformly, whereas the comparable standardized segments from the normal subject, as shown in FIG. 8, appear to thicken uniformly at the same time early in systole. Thus, as shown in FIG. 9, more variability in the time-to-maximal wall thickness of the standardized segments may indicate a greater degree of dyssynchrony.

Thus, the above-described system and method allows a clinician to use CT-acquired data to non-invasively determine the optimal site of LV lead placement for CRT by the co-registration of anatomic (coronary veins) and functional (LV dyssynchrony and myocardial scar) data to target regions of most delayed activation and avoid regions of myocardial scar. The present invention provides functional analytics that provide a non-invasive way to simulate EAM using regional myocardial velocity and acceleration, as well as a quantitative CT method for automatically deriving and analyzing global LV dyssynchrony using changes in wall thickness over time to predict CRT response.

The present invention has been described in terms of one or more preferred embodiments, and it should be appreciated that many equivalents, alternatives, variations, and modifications, aside from those expressly stated, are possible and within the scope of the invention.

1. A system for determining a pattern of activation of a heart of a subject, the system comprising:
a memory having stored thereon an imaging dataset acquired from a portion of the subject including the heart;
a processor having access to the memory and the imaging dataset stored thereon and configured to process the imaging dataset to identify a motion parameter and map the motion parameter over time to create a pattern of activation of the heart of the subject over time; and
a display coupled to the processor and configured to display the pattern of activation of the heart in a series of images of the heart of the subject over time.

2. The system as recited in claim 1 wherein the processor processes the imaging dataset to determining a systole and a diastole phase of a cardiac cycle.

3. The system as recited in claim 1 wherein the processor segregates the imaging dataset into a cardiac anatomy dataset and a motion dataset.

4. The system as recited in claim 3 wherein the processor merges the cardiac anatomy dataset and the motion dataset to form a combined dataset.

5. The system as recited in claim 1 wherein the processor processes the motion parameter using a non-rigid registration based algorithm to track a voxel-to-voxel movement during a cardiac cycle.

6. The system of claim 5 wherein the motion parameter includes velocity and the voxel-to-voxel movement is expressed as velocity.

7. The system as recited in claim 1 wherein the motion parameter includes a time-to-first-peak systolic velocity parameter.

8. The system as recited in claim 7 wherein the time-to-first-peak systolic velocity parameter is shown on the display using a binary color template.

9. The system as recited in claim 8 wherein the binary color template matches to an electroanatomical map (EAM) activation pattern color template.

10. The system as recited in claim 8 wherein the binary color template includes a first color and a second color such that myocardial regions of the heart are represented by the first color until a first upslope curve in a cardiac cycle is reached after which the activated myocardial regions of the heart are represented by the second color.

11. The system as recited in claim 1 wherein the display shows at least one of a series of images and a video.

12. The system as recited in claim 1 wherein the display identifies a site of latest activation to guide a left ventricular lead placement.

13. The system as recited in claim 1 wherein the processor is configured to generate a metric for a dyssynchrony index using changes in wall thickness of the heart of the subject over time.

14. The system as recited in claim 13 wherein the metric for the dyssynchrony index is an average of standard deviations (SD) of times to a maximal wall thickness of standardized segments of the heart.

15. The system as recited in claim 14 wherein variability in the times to maximal wall thickness of each standardized segment of the heart indicates a greater degree of dyssynchrony and uniformity in the times to maximal wall thickness of each standardized segment of the heart indicates a lesser degree of dyssynchrony.

16. A method for determining a pattern of electro-mechanical activation of a heart of a subject, the method comprising the steps of:
a) acquiring an imaging dataset from a portion of the subject including the heart;
b) segregating the imaging dataset into a cardiac anatomy dataset and a motion dataset;
c) processing the cardiac anatomy dataset to identify a cardiac phase of the heart over time;
d) processing the motion dataset to identify a motion parameter to operate as a surrogate for electrical activation;
e) merging the cardiac anatomy dataset and the motion dataset to form a combined dataset; and
generating a report related to the pattern of electro-mechanical activation of the heart of the subject using the combined dataset.

17. The method as recited in claim 16 wherein processing the cardiac anatomy dataset includes determining a systole and a diastole phase of a cardiac cycle.

18. The method as recited in claim 16 wherein processing the motion dataset includes using a non-rigid registration based algorithm to track a voxel-to-voxel movement during a cardiac cycle.

19. The method of claim 18 wherein the motion parameter includes velocity and the voxel-to-voxel movement is expressed as velocity.

20. The method as recited in claim 16 wherein the motion parameter of step d) includes a time-to-first-peak systolic velocity parameter.

21. The method as recited in claim 20 further comprising displaying the time-to-first-peak systolic velocity parameter in the report using a binary color template.

22. The method as recited in claim 21 further comprising matching the binary color template to an electroanatomical map (EAM) activation pattern color template.

23. The method as recited in claim 21 further comprising representing myocardia regions of the heart using a first color of the binary color template and a second color of the binary template, wherein myocardial regions of the heart are represented by the first color until a first upslope curve in the cardiac cycle is reached after which the activated myocardial regions of the heart are represented by the second color.

24. The method as recited in claim 16 further including segmenting the motion dataset using the anatomy dataset to localize the heart following step e).

25. The method as recited in claim 16 wherein the report includes at least one of a series of images and a video.

26. The method as recited in claim 16 wherein the report identifies a site of latest activation to guide a left ventricular lead placement.

27. The method as recited in claim 16 wherein the imaging dataset is a CT dataset.

28. The method as recited in claim 16 further comprising generating a metric for a dyssynchrony index using changes in wall thickness of the heart of the subject over time.

29. The method as recited in claim 28 further comprising calculating the metric for the dyssynchrony index by averaging standard deviations (SD) of times to a maximal wall thickness of standardized segments of the heart.

30. The method as recited in claim 29 further comprising indicating a degree of dyssynchrony when there is variability in the times to maximal wall thickness of each standardized segment of the heart and indicating a lesser degree of dyssynchrony when there is uniformity in the times to maximal wall thickness of each standardized segment of the heart.

31. A method for determining a pattern of activation of a heart of a subject, the method comprising the steps of:
a) acquiring an imaging dataset from a portion of the subject including the heart;
b) processing the imaging dataset to identify at least one motion parameter of the heart;
c) mapping the motion parameter over time to create a pattern of activation of the heart of the subject over time; and
d) displaying the pattern of activation of the heart of the subject over time.

32. The method of claim 25 wherein the imaging dataset includes at least a computed tomography (CT) dataset.
